(19) United States
(12) Patent Application Publication
(10) Pub. No.: US 2003/0208214 A1
(43) Pub. Date: Nov. 6, 2003

(54) Anastomotic connector and graft expander for mounting a graft

(76) Inventors: Amir Loshakove, Moshav-Bazra (IL); Ido Kilemnik, Herzelia (IL); Tanchum Feld, D N Izrael (IL); Dvir Keren, Petach-Tikva (IL)

Correspondence Address: Reed Smith, 29th Floor, 599 Lexington Avenue, New York, NY, 10022-7650, US

(21) Appl. No.: 10/239,365

(22) PCT Filed: Jan. 25, 2001
(86) PCT No.: PCT/IL01/00074

(51) Int. Cl. A61B 17/08

(52) U.S. Cl. 606/153

(57) ABSTRACT

An anastomotic connector device (102) for mounting a graft (100) in which the connector has spikes (106) defining a periphery around the base ring (104). Also a graft expander (900) having a body (906), a lower diameter tube portion (908) connected by truncated cone (910) and a finger rest (902) and plunger (904).

### RELATED APPLICATIONS

This application claims the benefit under 119(e) of U.S. provisional application 60/254,689. This application is a continuation in part of PCT applications PCT/EL99/00284, PCT/IL99/00670, PCT/IB00/00310, PCT/IL00/00611, PCT/IL00/00609 and a PCT application filed by same applicant on Jan. 24, 2001 in the Israel receiving office, entitled “GRAFT DELIVERY SYSTEM” and having serial number PCT/IL01/00069. The disclosures of all of these applications, which are filed by applicant Bypass and designate the US, are incorporated herein by reference.

### FIELD OF THE INVENTION

The present invention relates to anastomotic connectors and methods of mounting grafts on such connectors.

### BACKGROUND OF THE INVENTION

Anastomotic connections between two blood vessels are generally made by suturing or by mounting a dedicated connector on the edges of the two blood vessels. Some dedicated connectors include spikes, on which the edge of the blood vessel (known in general as a graft) is transfixed. Often, it is desirable to evert the end of the vessel over the connector, as well. Such transfixing and everting are generally difficult and time consuming manual tasks.

### SUMMARY OF THE INVENTION

An aspect of some embodiments of the invention relates to various methods of mounting a graft on an anastomotic connector. An exemplary connector on which to perform such mounting, includes a plurality of hook-tipped spikes that define a general shape of a cone, with a ring that interconnects the spikes at the base of the cone and the hooks, which point outwards and downwards at the apex of the cone. Optionally, these hooked ends are used for engaging a target vessel and/or retracting the target vessel relative to the graft. In some embodiments of the invention, instead of a hook shape, other geometrical shapes adapted to engage a blood vessel are used, for example, a bent fork. Additionally, the following methods may be applied to other connector designs as well.

In some exemplary embodiments of the invention, the connector is not substantially distorted by or as a result of the eversion process.

In one exemplary embodiment of the invention, the graft is brought through the connector ring and the apex of the connector and folded back over the hooks. The graft is pulled back sharply, causing the folded-back part of the graft to catch on the hooks and be transfixed on them.

In an alternative exemplary embodiment of the invention, the spikes of a connector are distorted by being folded over inwards to the inside of the cone, so that the hooks point inwards, leaving a space between them for the graft. Optionally, the hooks are held in place (e.g., by a dedicated spike holder). The graft is placed between the hooks and the hooks are released or advanced inwards towards the graft, to transfix it. Optionally, an inner mandrel is provided in the lumen of the graft, for example, to limit the advance of the hooks after they transfix the graft. The mandrel may then be removed and the spikes unbent.

In an alternative exemplary embodiment of the invention the spikes are distorted to be twisted around their axis, so that the hooks point in. In the mounting process, the graft is placed between the hooks and a piercable balloon (or other expandable element) is expanded within the graft to urge it against the hooks to be transfixed. The balloon is then removed.

An aspect of some embodiments of the invention relates to a method of twisting the spikes of an anastomotic connector, in which the spikes are held between an inner ring and an outer ring. At least one of the rings is pliable and is able to engage the spikes, for example, at least one of the rings is rough and/or has a high coefficient of friction. In one example, one or both of the rings comprising rubber rings. In use, one of the rings is rotated around the other, causing each individual spike to be twisted.

An aspect of some embodiments of the invention relates to transfixing a graft by a connector, using a piercable element. In an exemplary embodiment of the invention, the graft is placed between the spikes of the connector and the piercable element and then the element is impaled on the spikes, incidentally transfixing the graft on the spikes. Optionally, the element expands and in doing so, it optionally partially everts the lips of the graft.

An aspect of some embodiments of the invention relates to a method of transfixing a graft on a connector. The connector initially comprises substantially straight spikes and the tips of the spikes are bent into hooks after the graft is impaled on the spikes. These hooks are optionally suitable for engaging a target vessel at a later time. In some embodiments of the invention, the connector is placed on the graft so that it encircles the graft and the spikes are advanced relative to the graft, optionally after increasing the diameter of the graft, so that the spikes transfix the graft from the outside in. The tips of the spikes are then bent to form hooks. Optionally, the spikes are inclined inwards at an angle, to define a cone.

An aspect of some embodiments of the invention relates to a tool for graft inflating and/or everting. In an exemplary embodiment of the invention, the tool comprises two coaxial tubes of different diameters interconnected by a truncated cone. The graft is mounted on the smaller diameter tube and up the cone. The cone and tubes are then radially expanded and the end of the graft is rolled back into an everted position. In an exemplary embodiment of the invention, the cone and tube are made of a distortable material and are expanded by urging a plug into the tube.

An aspect of some embodiments of the invention relates to a method of mounting a graft on a connector, such that graft parts that are folded back lie between and adjacent forward spikes, rather than being transfixed by the spikes. The parts of the graft may be held in place by removable spikes while an anastomosis to a target vessel is being performed. Alternatively, the connector may include a second set of spikes, which hold the graft parts in place.

An aspect of some embodiments of the invention relates to a method of reducing an axial profile of an anastomotic connector. In an exemplary embodiment of the invention, spikes of the connector are tom at a point near the anastomosis connection, by pulling back on the spikes. Optionally, a weakening is formed at the point, to guide the tearing.

An aspect of some embodiments of the invention relates to a gauge and method for measuring a graft for an anastomosis connection. In an exemplary embodiment of the invention, the gauge comprises a tube having a known diameter. An attempt is made to evert the graft over the tube. If the attempt succeeds, a system that matches the gauge (e.g., color coding) is selected for use. If the attempt fails, a different size tube is tried or a larger or smaller connector and/or delivery system size is used.

There is thus provided in accordance with an exemplary embodiment of the invention a method of mounting a graft on a spiked connector, in which the tips of the spikes define a periphery, comprising:

placing the graft, in said periphery, between tips of said spikes and a piercable element;

impaling said piercable element on said spikes, such that the graft is also impaled on said spikes; and

removing said piercable element from said spikes. Optionally, the method comprises pulling back impaled parts of said graft to a side of the spikes opposite said tips. Alternatively or additionally, said piercable element is expandable, the method comprising inflating said expandable element. Optionally, said expansion impales said element on said spikes. Alternatively or additionally, said expansion at least partially everts said graft.

In an exemplary embodiment of the invention, said impaling comprises advancing said spike tips towards said graft. Alternatively or additionally, said impaling comprises releasing said spike tips to advance towards said graft.

In an exemplary embodiment of the invention, the method comprises bending said spike tips into hooks after said impaling. Alternatively or additionally, the method comprises forming said spike tips into hooks before said impaling.

There is also provided in accordance with an exemplary embodiment of the invention, a method of mounting a graft on a spiked connector, comprising:

placing the graft between tips of said spikes and an element having a general outer perimeter;

advancing said spikes relative to said element such that said spikes penetrate said graft and penetrate said perimeter; and

removing said element.

Optionally, said element is formed of a hard material and includes a plurality of depressions to receive said spikes. Optionally, said spikes are pre-bent and wherein said depressions guide an unbending of said spikes.

In an exemplary embodiment of the invention, said element is piercable by said spikes. Optionally, said element is expandable.

In an exemplary embodiment of the invention, said element is non-expandable.

In an exemplary embodiment of the invention, advancing said spikes comprises advancing spikes that are adapted for engaging a target vessel of the anastomosis.

There is also provided in accordance with an exemplary embodiment of the invention, a method of mounting a graft on a connector having a plurality of spikes arranged around a central opening, said spikes having radially outward pointing hooks, comprising:

folding said spikes such that said hooks point inward into said central opening and define a periphery between them;

inserting a graft into said periphery, advancing said hooks relative to said graft, to penetrate said graft; and

repositioning said hooks to point outward. Optionally, advancing said hooks comprises releasing said spikes. Alternatively or additionally, advancing said hooks comprises moving said spikes. Alternatively or additionally, advancing said hooks relative to said graft comprises radially expanding said graft towards said hooks. Alternatively or additionally, the method comprises inserting a contra mandrel in said graft, to limit an advance of said hooks. Optionally, said contra mandrel guides said repositioning.

In an exemplary embodiment of the invention, repositioning said hooks comprises unfolding said spikes.

There is also provided in accordance with an exemplary embodiment of the invention, a method of mounting a graft on a connector having a plurality of spikes arranged around a central opening, said spikes having radially outward pointing hooks, comprising:

inserting a graft into a periphery defined by forward ends of said spikes, in a first direction;

folding back a tip of said graft to cover said hooks; and

pulling back said graft, in a direction opposite said first direction, such that said hooks engage said folded part of said graft. Optionally, pulling back said graft comprises sharply pulling on said graft. Alternatively or additionally, the method comprises advancing said engaged part of said graft towards a base of said spikes, in said opposite direction.

In an exemplary embodiment of the invention, said spikes define a cone, with said hooks at an apex of said cone.

There is also provided in accordance with an exemplary embodiment of the invention, a method of forming spike tips of an anastomotic connector, into hooks for engaging blood vessels, comprising:

providing an anastomosis connector having a plurality of straight spikes having tips;

first bending said tips at a first angle, using a first mandrel; and

second bending a furtherly distal portion of said tips using a second mandrel, to form a hook shape of said tips. Optionally, the method comprises mounting a graft on said spikes of said connector prior to said first bending.

In an exemplary embodiment of the invention, said first and said second mandrel have different outside diameters.

There is also provided in accordance with an exemplary embodiment of the invention, an anastomotic connector, comprising:

a base ring; and

a plurality of hooked spikes that pass through said base ring, which spikes include a weakening adjacent said hooks,

wherein a position of said weakening on said spikes is located on said spike so that the spikes can be torn off at said weakening by retracting said spikes relative to said hooks. Optionally, said spikes include an extension perpendicular to said spike and distal of said weakening. Optionally, said extension has an extent greater than apertures in said base ring through which said spikes pass. Alternatively or additionally, said extension is adapted to serve as a base for holding said hooks, when tearing said spikes.

In an exemplary embodiment of the invention, said spike have tips that are pre-stressed to bend when said spike is torn. In an exemplary embodiment of the invention, said extension serves as a ratchet mechanism. Alternatively or additionally, said base ring comprises a plurality of apertures for said spikes. Optionally, the apertures include a spring formed of said base ring.

There is also provided in accordance with an exemplary embodiment of the invention, an anastomotic connector, comprising:

a base ring; and

a plurality of target spikes, adapted to engage a target vessel, which target spikes pass through said ring; and

a plurality of retractable pulling spikes, having tips adapted to engage a graft placed in the lumen of said ring, and being adapted to at least partially evert a lip of said graft when said pulling spikes are retracted. Optionally, said target spikes are hooked. Alternatively or additionally, said target spikes are inclined towards an axis of said connector. Alternatively or additionally, said puller spikes are provided through said base ring. Alternatively or additionally, said puller spikes are provided through a ring other than said base ring.

In an exemplary embodiment of the invention, said puller spikes include at least one weakened location, to facilitate tearing of said spikes. Alternatively or additionally, said puller spikes are adapted to be straightened by said ring, when they are retracted.

There is also provided in accordance with an exemplary embodiment of the invention, a method of performing an anastomosis, comprising:

engaging at least one of the vessels of a two vessel anastomosis, using a plurality of retractable spikes;

retracting said plurality of retractable spikes, to cause at least a partial eversion of said vessel. Optionally, the method comprises, completing said anastomosis. Alternatively or additionally, the method comprises, removing said retractable spikes.

There is also provided in accordance with an exemplary embodiment of the invention, a method of partially everting a graft on a connector, comprising:

inserting a graft into a ring shaped anastomosis connector having a plurality of spikes; and

pulling an end of said graft radially out, so that said end abuts said spikes adjacent the spikes and extends radially out of said spikes between said spikes. Optionally, said pulling comprises pulling using retractable spikes.

There is also provided in accordance with an exemplary embodiment of the invention, a method of twisting hook-tipped spikes in a ring-type anastomotic connector so that the hooks point inward rather than outward, comprising:

arranging said spikes between two rings; and

rotating one of said rings relative to the other of said rings such that said spikes are twisted at least 120°. Optionally, at least one of said rings comprises a rubber ring. Alternatively or additionally, the method comprises holding said twisted spikes in a twisted configuration, during an eversion process.

There is also provided in accordance with an exemplary embodiment of the invention, a method of selecting an anastomotic connector for a graft, comprising:

test everting a graft on a hollow, tubular gauge having a gauge diameter; and

if said everting succeeds, selecting a connector having a size that matches said gauge diameter. Optionally, said selected connector is pre-loaded in a delivery system, in a sterile package. Optionally, said package is color-coded to match said gauge.

In an exemplary embodiment of the invention, test everting comprises comprising selecting first a larger gauge for test-everting. Alternatively or additionally, test everting comprises selecting first a smaller gauge for test-everting, first.

There is also provided in accordance with an exemplary embodiment of the invention, a medical graft expander, comprising:

a body having a first diameter;

a tube having a second diameter smaller than said first diameter, mounted at an end of said body, and defining an inner chamber; and

a shaft, positioned in said body and axially advancable into said chamber, said shaft having at least one section with a diameter greater than a diameter of said chamber, such that when said section is advanced into said chamber, said tube is radially expanded. Optionally, said tube is made of a soft material. Optionally, said tube is made of silicone.

In an exemplary embodiment of the invention, said tube is sized to be larger than an inner diameter of a graft, when the tube is expanded. Alternatively, said tube is sized to match an inner diameter of a graft, when the tube is expanded.

There is also provided in accordance with an exemplary embodiment of the invention, a graft everting method, comprising:

mounting at least an end of said graft on an expandable tube;

expanding said tube to engage and expand said graft; and

rolling back at least a portion of said end over itself Optionally, the method comprises providing a tube over said graft, so said rolling back is on to said tube. Alternatively or additionally, said tube is pre-loaded with an anastomotic connector.

### BRIEF DESCRIPTION OF THE FIGURES

Non-limiting embodiments of the invention will be described with reference to the following description of exemplary embodiments, in conjunction with the figures. The figures are generally not shown to scale and any measurements are only meant to be exemplary and not necessarily limiting. In the figures, identical structures, elements or parts which appear in more than one figure are preferably labeled with a same or similar number in all the figures in which they appear, in which:

FIGS. 1A-1C illustrate a method of everting a graft over an anastomosis connector, in accordance with an exemplary embodiment of the invention, in which the connector is not distorted;

FIGS. 2A-2C illustrate a method of everting a graft over an anastomosis connector, in accordance with an exemplary embodiment of the invention, in which spikes of the connector are folded backwards;

FIGS. 3A-3D illustrate a method of everting a graft over an anastomosis connector, in accordance with an exemplary embodiment of the invention, in which spikes of the connector are twisted;

FIGS. 4A-4C illustrate apparatus for use in the method shown in FIGS. 3A-3D, for maintaining the spikes in a twisted configuration, in accordance with an exemplary embodiment of the invention;

FIGS. 5A-5I illustrate a method of everting a graft over an anastomosis connector, in accordance with an exemplary embodiment of the invention, in which spikes of the connector are formed into hooks after the graft is penetrated;

FIGS. 6A and 6B illustrate a variation of the method of FIGS. 5A-5I;

FIGS. 7A-7C illustrate apparatus for bending the tips of the spikes, for the method of FIGS. 5 and 6, in accordance with an exemplary embodiment of the invention;

FIGS. 8A-8C illustrate an alternative apparatus for bending the tips of the spikes, for the method of FIGS. 5 and 6, in accordance with an exemplary embodiment of the invention;

FIGS. 9A-9C illustrate a graft expander, in accordance with an exemplary embodiment of the invention;

FIG. 10 illustrates a graft gauge, in accordance with an exemplary embodiment of the invention;

FIGS. 11A-11E illustrate a connector in which a partial eversion is achieved, in accordance with an exemplary embodiment of the invention;

FIGS. 12A illustrates a part of an anastomotic connector, in accordance with an exemplary embodiment of the invention;

FIGS. 12B-12D illustrate a process of deploying a connector, in which part of the connector is removed; and

FIGS. 12E-12G illustrate the effect of the process of FIGS. 12B-12D, on a single spike of the connector.

### DETAILED DESCRIPTION OF EXEMPLARY EMBODIMENTS

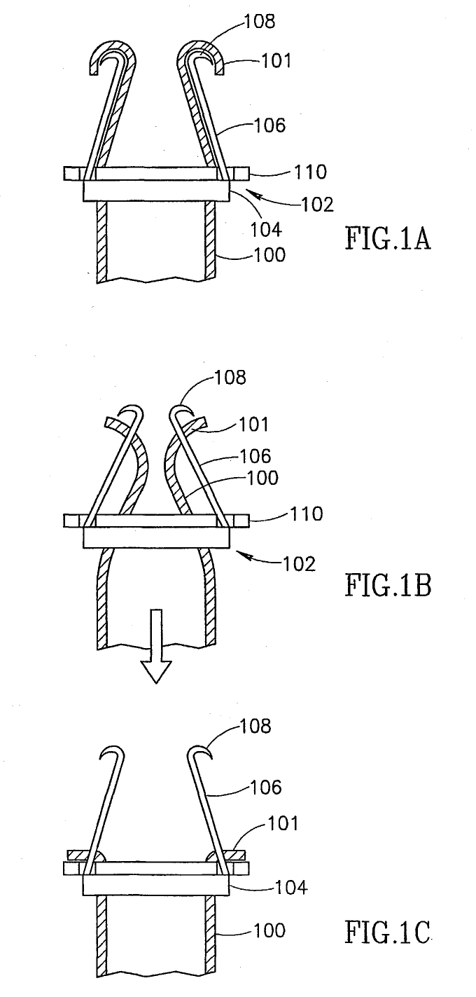

FIGS. 1A-1C illustrate a method of everting an end 101 of a graft 100 over an anastomosis connector 102, in accordance with an exemplary embodiment of the invention, in which connector 102 is not distorted.

Connector 102 comprises a ring 104 on which a plurality of spikes 106 are mounted. The tips of each of spikes 106 is bent back in the shape of a hook 108. Optionally, all spikes 106 of connector 102 are bent inwards to define a cone shape. In addition, connector 102 optionally includes a base ring 110. This is the exemplary connector design used in various of the embodiments described herein. However, as will be appreciated, many of the eversion methods do not require the connector to be of the above type. For example, the connector may be a one-part connector, for example as described in the above related applications.

In FIG. 1A, graft 100 is brought through ring 104 and between hooks 108 of spikes 106. End 101 of graft 100 is everted manually over hooks 108. It should be noted that this eversion is relatively easy to perform, as the outer diameter of the hooks is significantly smaller than that of ring 104 on which the graft is everted at the end of the process.

In FIG. 1B, graft 100 is pulled back sharply, so that the tips of hooks 108 pierce end 101, thus transfixing graft 100 on spikes 106.

In FIG. 1C, graft 100 is pulled back slowly (or end 101 pushed down) so that graft 100 is evereted over ring 104.

It should be noted that this and other eversion methods described below may be performed on attached or unattached (to the body) grafts and inside or outside of the body. In addition, although a perpendicular anastomotic device is shown, in some exemplary embodiments of the invention, an oblique connection device is provided and/or an oblique eversion is performed. For example, an oblique connector can have spikes or hooks of unequal lengths and/or an axis not perpendicular to ring 104. Alternatively or additionally, in some embodiments, graft 100 is provided at an angle to the axis of connector 102.

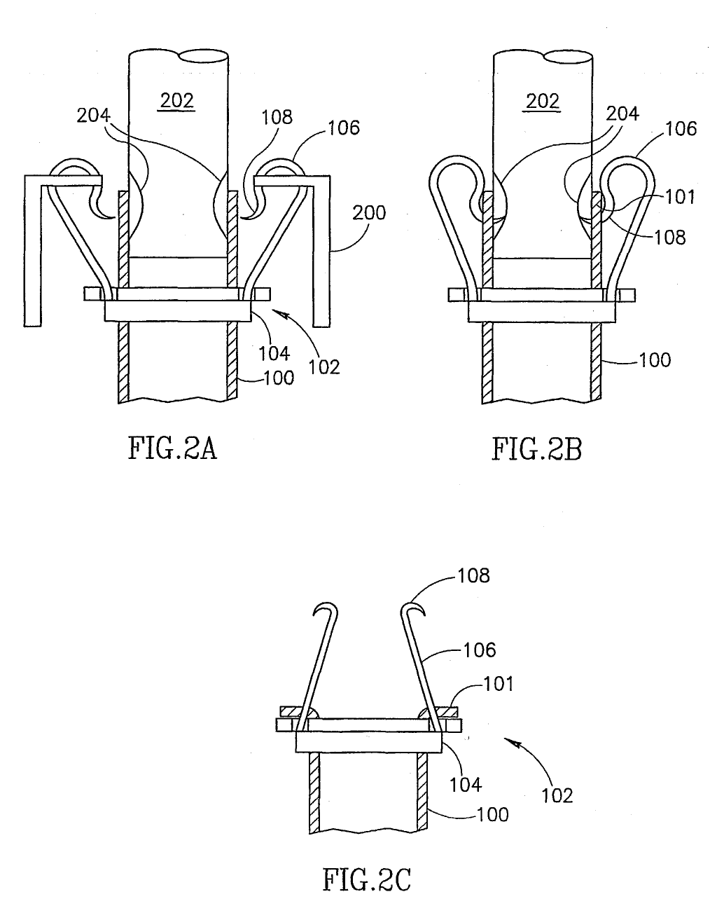

FIGS. 2A-2C illustrate a method of everting graft 100 over anastomosis connector 102, in accordance with an exemplary embodiment of the invention, in which spikes 106 are folded to reorient the hook direction.

In FIG. 2A, a holder 200, holds spikes 106 so that they are folded back and the inner diameter between hooks 108 is large enough that graft 100 can fit between them. Hooks 108 are pointed to be generally perpendicular to graft 100. A contra mandrel 202 is optionally provided inside graft 100.

In FIG. 2B, holder 200 releases spikes 102 so that hooks 108 penetrate graft 100 at end 101. Contra 202 can prevent hooks 108 from advancing too far, optionally receiving the hooks in depressions 204 in contra mandrel 202. Possibly, holder 200 (not shown) still engages the spikes to prevent them from advancing too far (e.g., in addition to or instead of contra mandrel 202) or to prevent them from unfolding in a wrong direction.

In an exemplary embodiment of the invention, holder 200 comprises a plurality of tweezers mounted on a base, each one of which tweezers holds one spike.

In FIG. 2C, contra mandrel 202 is optionally removed and spikes 106 are allowed to unfold. Alternatively or additionally, contra mandrel 202 remains, for example, to assist in directing the unfolding of the spikes. End 101 is then pulled down towards ring 104.

Although a same connector as in FIG. 1 may be used, optionally, the connector of FIG. 2 has longer spikes.

In an exemplary embodiment of the invention, spikes 106 are folded back by applying pressure to the spikes towards base ring 104, for example, using a pressure ring, at a point adjacent the tips of the hooks.

The above description of FIG. 2 assumed that the connector is super-elastic, elastic or has shape memory and even when distorted will revert to its earlier shape, when released. Alternatively, connector 102 is a plastically deformed connector. The transition from FIG. 2A to FIG. 2B to FIG. 2C may then be an active transition, with an operator manually bending and unfolding the spikes.

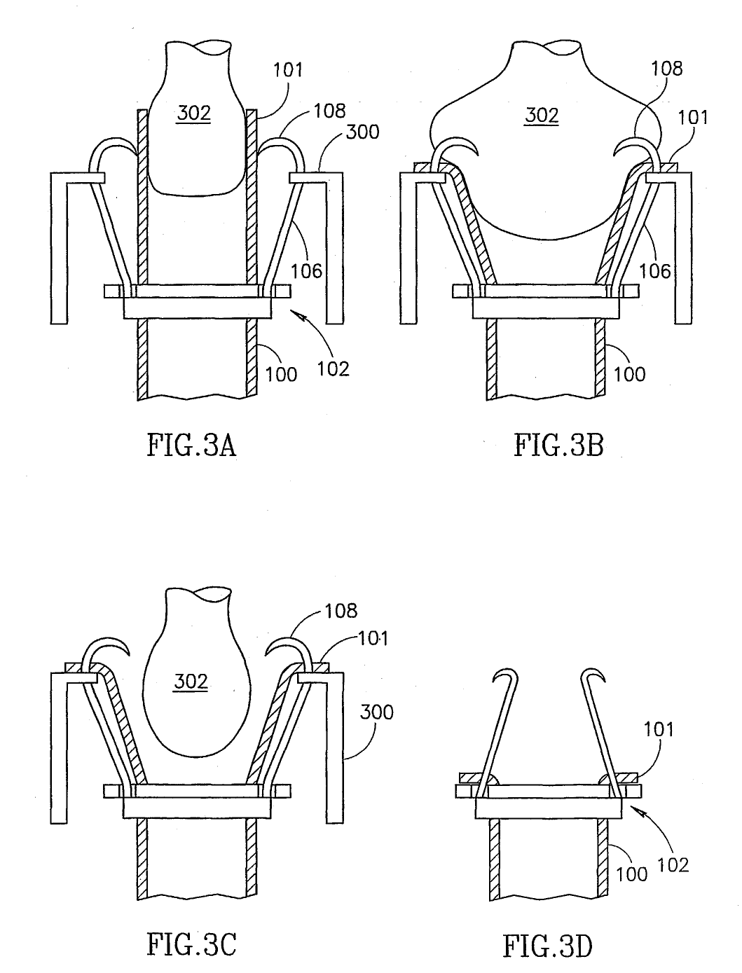

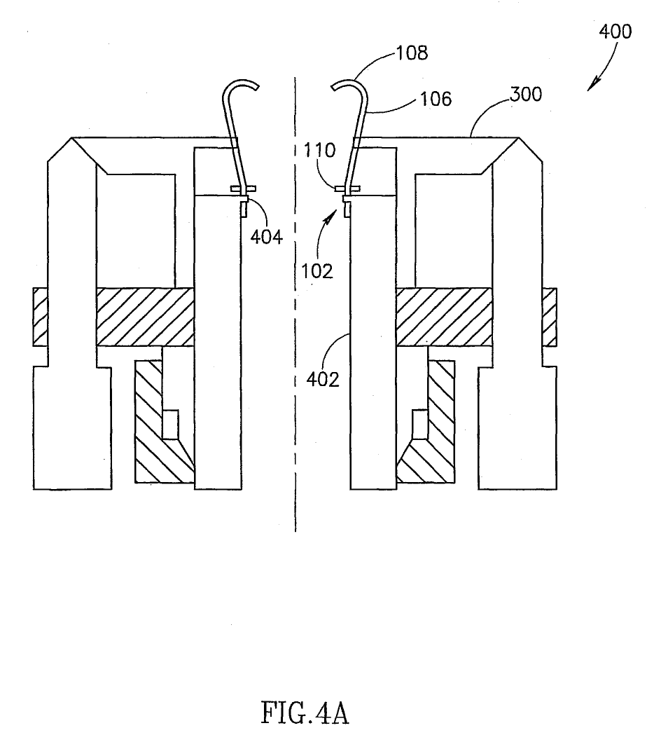

FIGS. 3A-3D illustrate a method of everting graft 100 over anastomosis connector 102, in accordance with an exemplary embodiment of the invention, in which spikes 106 are twisted.

FIG. 3A shows connector 102 being held by a spike holder 300, such that spikes 106 are twisted 180° on their main axis, so that hooks 108 point in, rather than out and so that a sufficient lumen is defined by the hook tips, to allow graft 100 to be passed between them. A piercable, optionally expandable, element 302 is placed inside the lumen of graft 100, and possibly inflated slightly, so that the graft is urged against hooks 108. Expandable element 302 may be, for example, a silicon bag with thick walls and a relatively small inner volume that can be filled with water. Alternatively, expandable element 302 may comprise a deformable framework.

In FIG. 3B, element 302 is expanded so that it becomes transfixed on the spikes. End 101 of graft 100, being between the hooks and pierced element 302 thus becomes transfixed by hooks 108.

In FIG. 3C, element 302 is deflated and/or spikes 106 are pulled away.

In FIG. 3D, the spikes are release (or deformed) and graft 100 is pulled down, so that the mounting is complete.

It should be noted that a contra mandrel 202 may be used in FIG. 3 instead of an expandable element and that an expendable element 302 may be used in FIGS. 2 instead of a contra mandrel 202. Both types of elements have the property that when graft end 101 is placed between the element and inward pointing hooks and either the graft is pushed out or the hooks pushed in, the hooks transfix the graft.

Alternatively to twisting spikes 106 before impaling the graft. In an alternative embodiment of the invention, spikes 106 are formed to have hooks 108 point inwards. The spikes are then permanently twisted to reposition the spikes outward. The spikes may be twisted prior to forming the hooks. Alternatively, the spikes, in the final connector are twisted, rather than flat. In some embodiments, no expandable or piercable element is used.

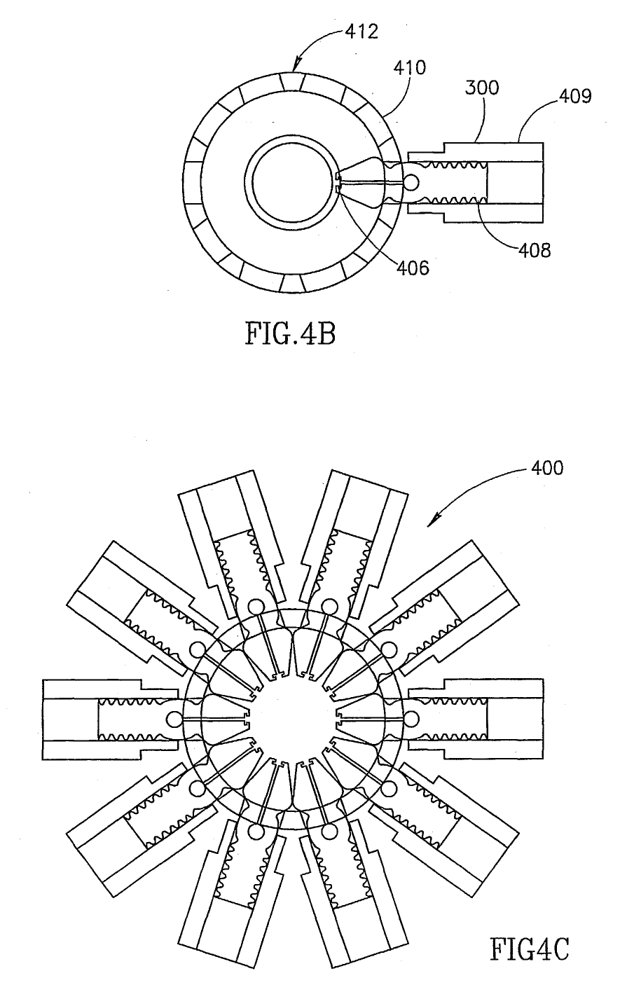

FIGS. 4A-4C illustrate apparatus 400 for use in the method shown in FIGS. 3A-3D, for maintaining spikes 106 in a twisted configuration, in accordance with an exemplary embodiment of the invention.

FIG. 4A shows connector 102 mounted on a delivery tube 402 and optionally restrained from rotation by a plurality of fingers 404 in the inner circumference of tube 402.

FIG. 4B shows a top view of apparatus 400 showing only a single spike holder 300. In FIG. 4C, all of holders 300 are shown. Referring to FIG. 4B, an exemplary spike holder 300 comprises a holding area 406 formed to hold a spike and prevent it from twisting back. As shown, the holding mechanism is based on a matching of the shape of area 406 to the profile of an exemplary spike (a rectangle). Alternatively, a force based mechanism, for example, a clamp, may be used. Holder 300 optionally comprises an external body 409 and an internal, optionally threaded, body 408 for adjusting the size of area 406 and/or its relative position. This may allow a single device to be used for multiple connector sizes, spike sizes and/or shapes and/or to compensate for manufacturing errors. Optionally the spikes are released by retracting outer body 409. Alternatively, the spikes may be removed manually.

A plurality of spike holders 300 may be mounted on a ring 412, each in a space 410 arranged to receive the holder. Other designs may be used as well.

In an exemplary embodiment of the invention, the spikes are twisted manually, for example using tweezers, prior to being placed in holding area 406. Optionally, the spike is twisted 270° and inserted into area 406 via their narrow profile. Then, when the spikes starts untwisting, it is locked into area 406.

In some exemplary embodiments of the invention, the spikes are twisted mechanically. In one example, the spikes are arranged between an inner and an outer rubber ring. When one of the rings is rotated around the connector axis, the spikes are twisted. Optionally, this process is applied when apparatus 400 is arranged near the spikes, for example, with areas 406 left open to allow spike rotation. Then when the spikes are rotated, areas 406 are minimized, to clamp down on the spikes.

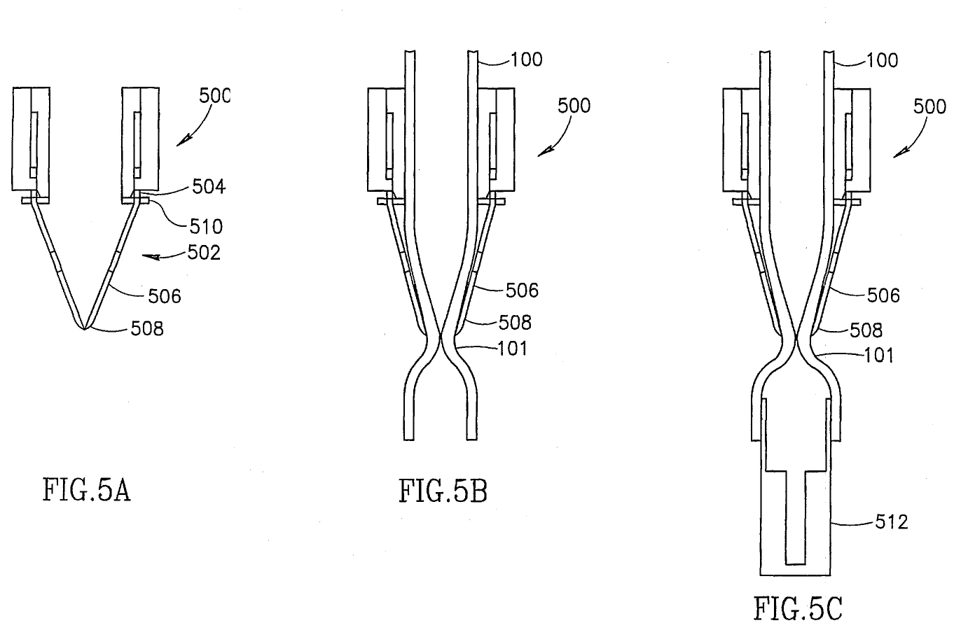

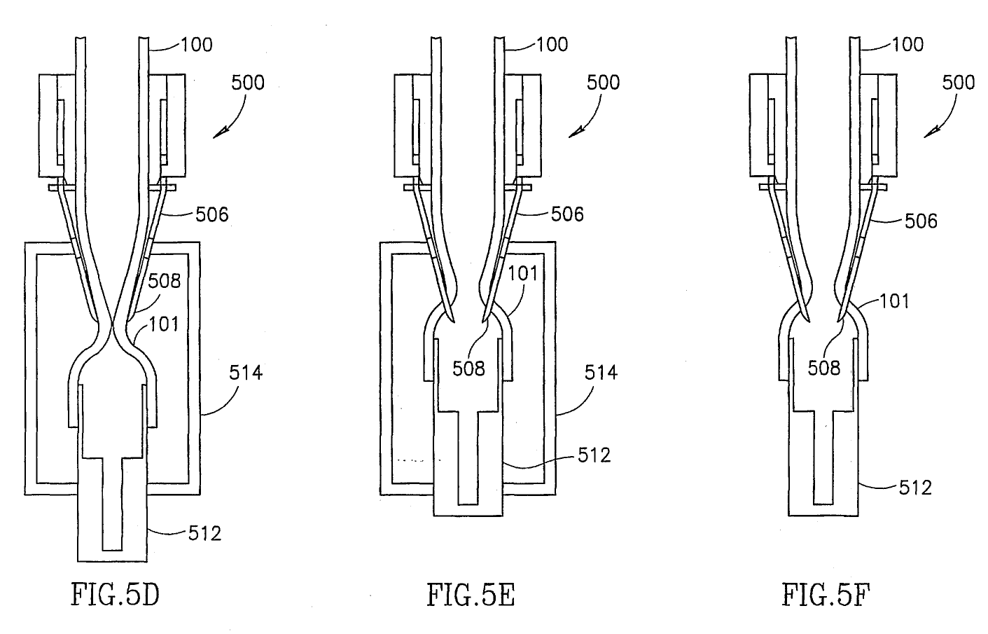

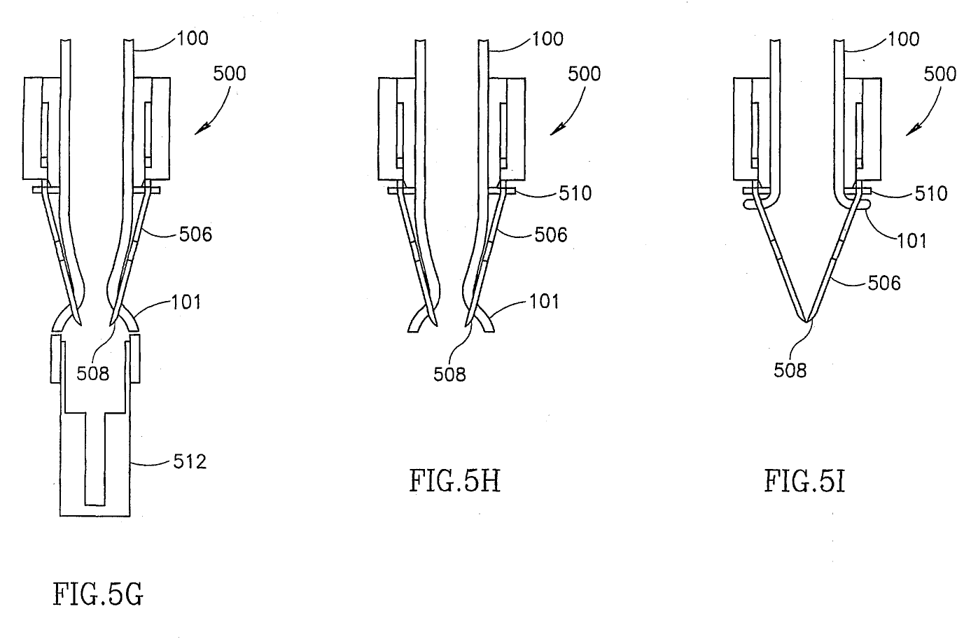

FIGS. 5A-5I illustrate a method of everting graft 100 over an anastomosis connector 502, in accordance with an exemplary embodiment of the invention, in which spikes 506 of the connector are formed into hooks after the graft is penetrated.

FIG. 5A shows connector 502 mounted on a connector holder 500. A plurality of inwardly inclined spikes 506, having straight tips 508 are shown. Ring 504 and base 510 correspond, in this exemplary connector, to ring 104 and base 110 of the previous figures.

In FIG. 5B, graft 100 is brought through connector holder 500 and between tips 508.

In FIG. 5C, graft 100 is shown to be mounted on a graft holder 512, which optionally ensures that the graft will have an inner diameter larger than the outer diameter of spike tips 508. Graft holder 512 can optionally expand.

In FIG. 5D, a spike restrainer 514 is shown, for preventing radial expansion of spikes 506.

In FIG. 5E, connector holder 500 is advanced relative to graft holder 512, such that spike tips 508 penetrate graft 100.

In FIG. 5F, spike restrainer 514 is removed.

In FIG. 5G, the graft is cut off near tips 508, to define graft end 101.

In FIG. 5H, graft holder 512 is removed.

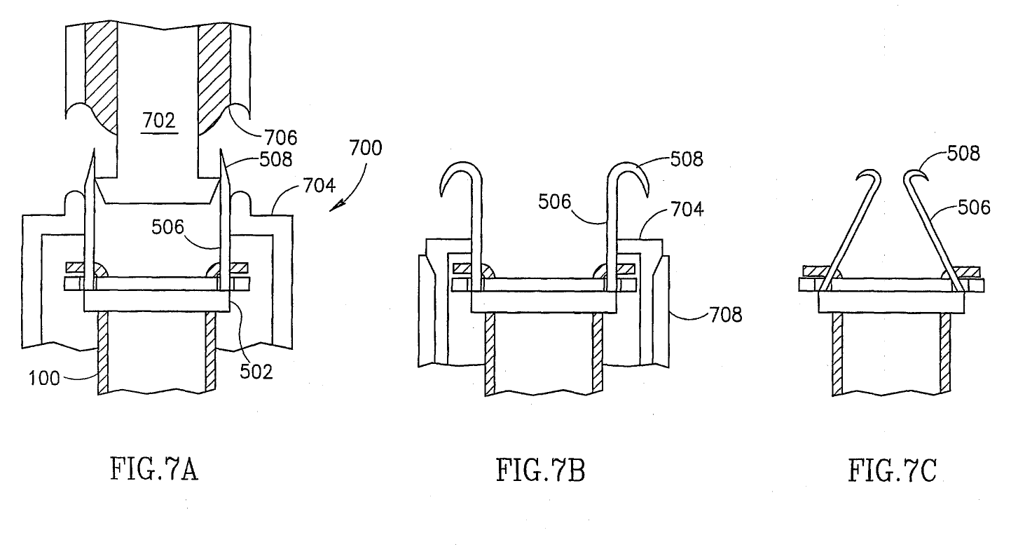

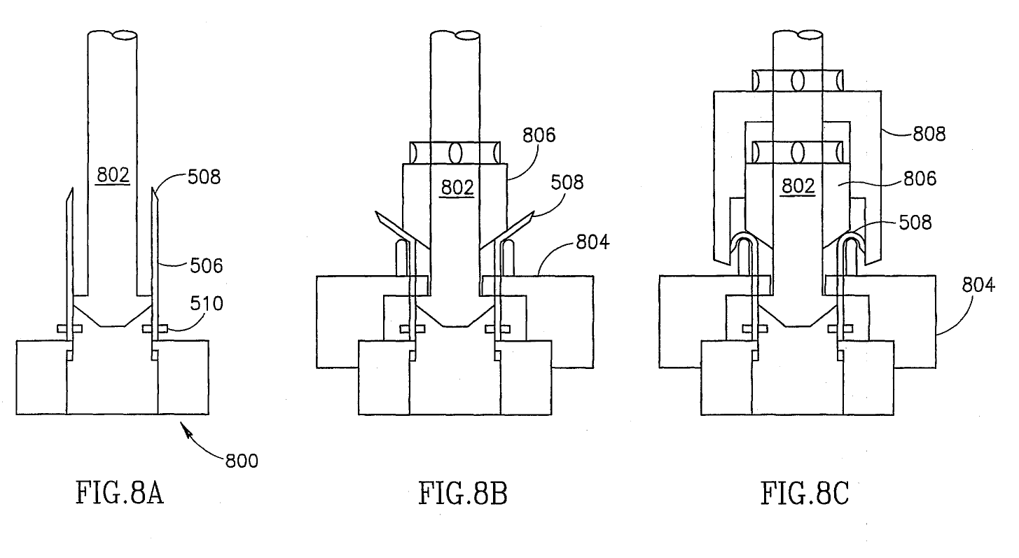

In FIG. 5L graft end 101 is pulled back towards ring 504, completing the mounting. Next, tips 508 are bent into hooks, as will be described in FIGS. 7 and 8.

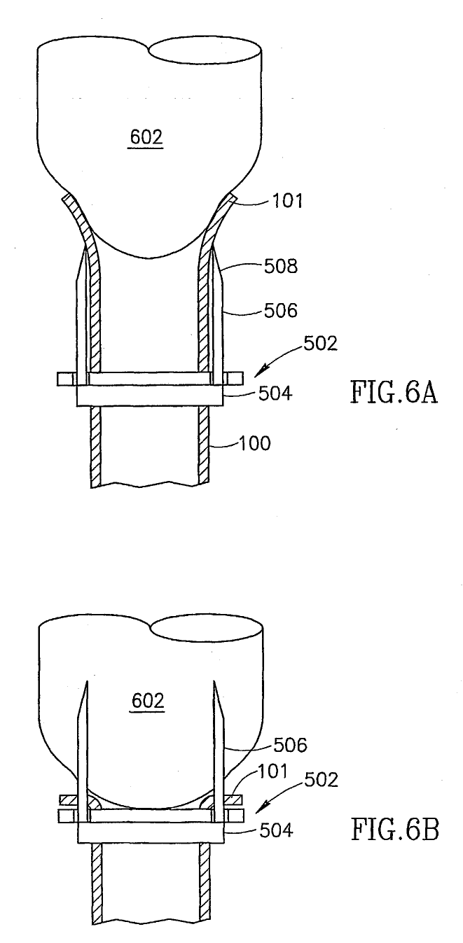

FIGS. 6A-6B illustrate a variation of the method of FIGS. 5A-5I, in which, instead of a graft holder, an expandable element 602 is provided. When element 602 is expanded and advanced, the graft is transfixed on spikes 506 and its end 101 is pushed back towards ring 504. Alternatively, element 602 is a non-expandable bulbous element, for example made of silicone rubber. Spikes 506 may be perpendicular to ring 504 as shown or they may be inwards inclined, and spread apart by the leading edge of element 602.

FIGS. 7A-7C illustrate apparatus 700 for bending the tips of spikes 506, for the method of FIGS. 5 and 6, in accordance with an exemplary embodiment of the invention.

In FIG. 7A, connector 502 is shown being held between spike holders 704 and an inner mandrel 702. Mandrel 702 has curved regions 706 formed therein for receiving spike tips 508 and, as mandrel 702 is advanced, bending them against a matching form on spike holders 704.

In FIG. 7B, spike tips 508 are bent into hooks and an overtube 708 is advanced to urge spike holders 704 inwards so that spikes 508 will be inwards bent, as shown in FIG. 7C.

Spikes 506 may be formed of a plastically deformable material. Alternatively, by providing a sharp enough bending angle (possibly with a considerable over-shoot), even elastic, super elastic or shape memory material will be permanently distorted into the shape of a hook. Alternatively or additionally, tips 508 are heat treated or otherwise processed, during manufacture or during use, for example, before, during or after the bending, to reduce or remove any shape memory or super-elastic property they may have.

FIGS. 8A-8C illustrate an alternative apparatus for bending tips 508, for the method of FIGS. 5 and 6, in accordance with an exemplary embodiment of the invention.

FIGS. 8A-C show a two step bending process. In FIG. 8A, connector 502 is held by a bending apparatus 800, with an inner mandrel 802. In FIG. 8B, a spike holder 804 is shown and a first bending mandrel 806 is brought down towards connector 502 to bend tips 508 a first bend. In FIG. 8C, A second bending mandrel 808 is brought down towards connector 502 to complete the bending of tips 508 into hooks.

In FIGS. 8A-8C, an alternative method (to FIG. 7) is shown for holding the spikes, in which spike holder 804 engages the spikes, for example by having a slot formed therein.

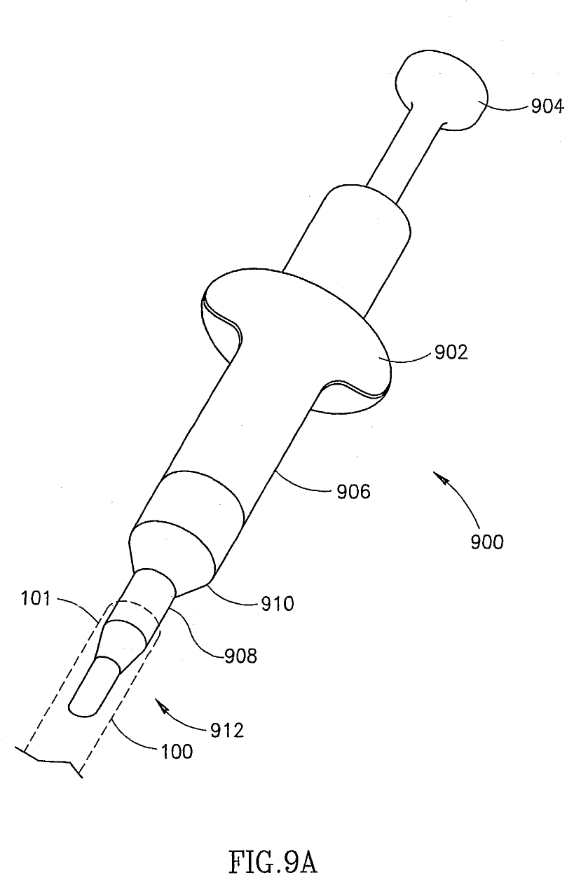

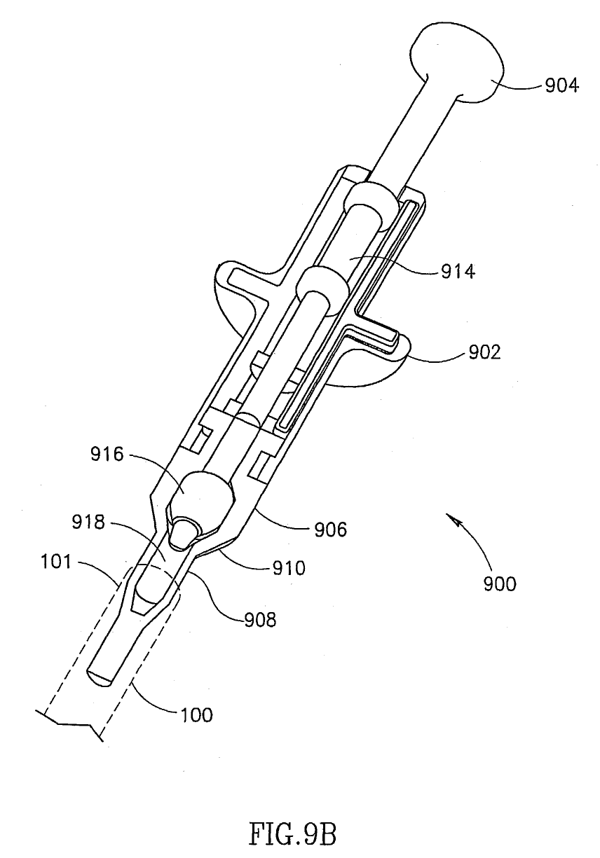

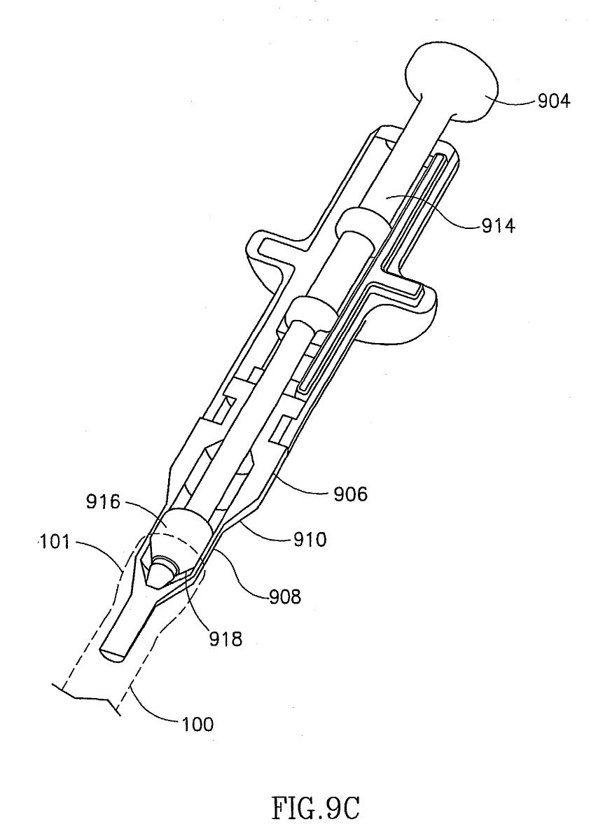

FIGS. 9A-9C illustrate a graft expander 900, in accordance with an exemplary embodiment of the invention. Expander 900 comprises a body 906 having, at its distal end, an optional tip portion 912 and a lower diameter tube portion 908 connected by an optional truncated cone 910 to body 906. Body 906 further comprises an optional finger rest 902 and a plunger 904 (or other means) for expanding tube portion 908. Also shown is graft 100 mounted on tube portion 908.

FIGS. 9B and 9C show a cut-through view of expander 900 in an unexpanded and an expanded configurations, showing plunger 904 including a shaft 914 having a widening 916 at its distal end, for effecting the expansion of tube portion 908.

In use, graft 100 is mounted onto lower diameter tube portion 908. When plunger 904 is advanced, widening 916 is forced into a chamber 918 in portion 908, causing tube portion 908 to expand in diameter and expand graft 100.

Graft 100 is then everted by rolling back its end 101, as shown in FIG. 9C, for example using fingers or tweezers. Possibly, the rolling back is performed directly onto a graft delivery system or onto a gauge (described below).

In an exemplary embodiment of the invention, the outside of expander 900 is formed of a soft material, such as silicone rubber. Plunger 904 and shaft 914 are possibly made of a harder material, such as Teflon. A hard-material stiffener may also be provided for finger rest 902.

In an oblique eversion device, the cross-section of tube portion 908, in its expanded form, may be elliptical. Alternatively or additionally, tube portion 908 bends when it is “expanded”, for example by providing stiffening wires in one side of the tube and/or by providing a tube with a radially non-uniform wall thickness. Optionally, the outer surface of tube portion 908 is made rough, to better engage the inner surface of graft 100, when expanded.

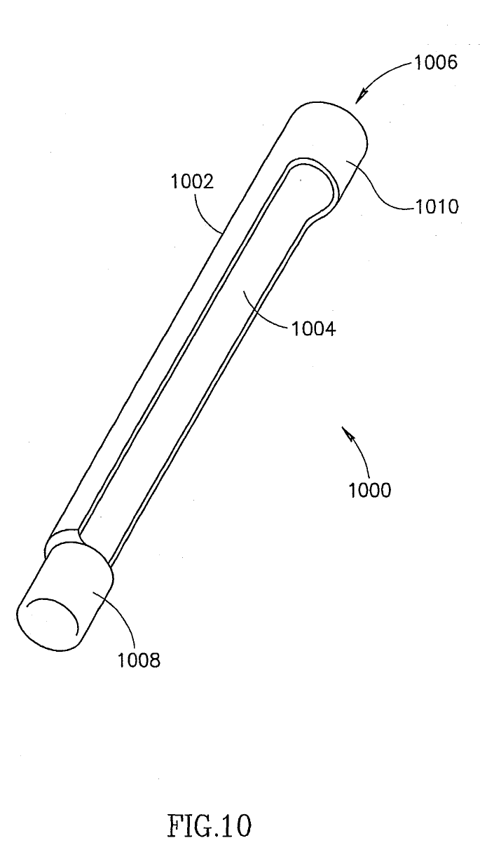

FIG. 10 illustrates a graft gauge 1000, in accordance with an exemplary embodiment of the invention.

Different graft sizes may require different sizes of connectors and/or different sizes of delivery systems. In some embodiments of the invention, the delivery systems are provided pre-loaded with a connector, inside a sterile packaging. Opening a package only to find that the delivery system does not fit the available graft, might be quite wasteful. In an exemplary embodiment of the invention, one or more separately packaged graft gauges are provided, for determining which of a plurality of available connector/delivery system sizes should be used for the graft. Graft expander 900 may also be matched to a single graft size. Alternatively, it may expand to multiple sizes, for example, by controlled expansion of tube 908. Optionally, widening 916 is conical (or step-wise), to allow for several expansion sizes.

Possibly, the gauges are color coded to match the delivery system packages. In an exemplary embodiment of the invention, two sizes of connectors are provided, small and large. If the large one is too large, the smaller one is used.

The exemplary gauge 1000 measures a graft by attempting to evert the graft on an end 1010 of the gauge. If the gauge is too large, the eversion attempt will fail. Graft expander 900 may be used to assist the eversion. In use, a graft is placed through an aperture 1004 in a tubular body 1002 of gauge 1000 and advanced out of an end-opening 1006 of gauge 1000. The other end of the gauges may be solid, for example comprising a handle 1008, or it may be a different diameter end, for performing a different measurement. Possibly, a single gauge comprises a more than two tubes that share a single aperture and have different diameter ends for attempting eversion. Alternatively or additionally, end 1010 has a graded diameter, for example having a sloped or step profile. Alternatively or additionally, end 1010 includes one or more inner tubes of lesser diameter, one which the eversion can be attempted first, and then a larger sized tube advanced into the everted region of the graft.

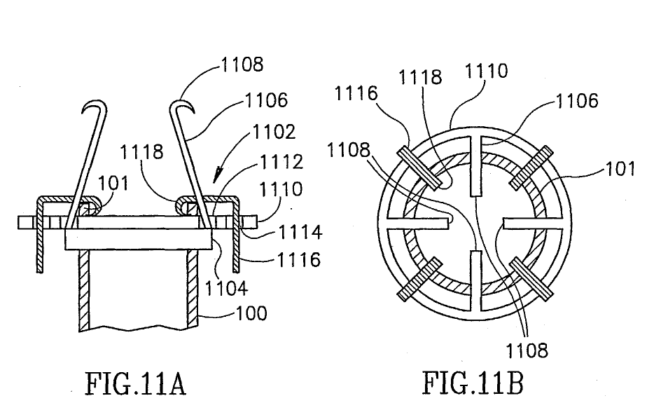

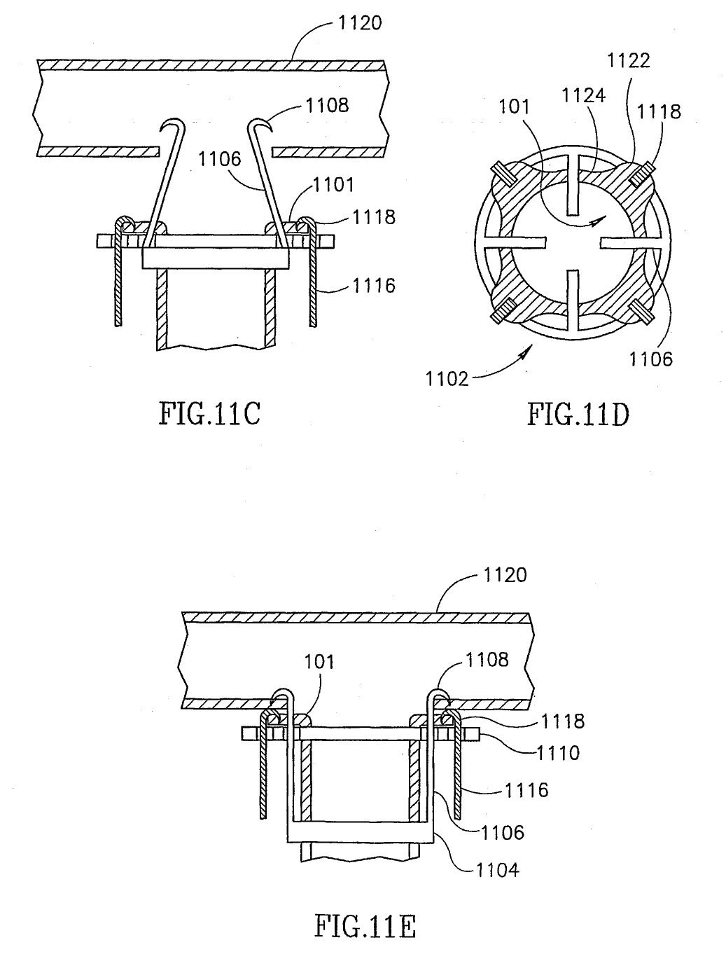

FIGS. 11A-11E illustrate a connector 1102 in which a partial eversion is achieved, in accordance with an exemplary embodiment of the invention. Connector 1102, is superficially similar to connector 102, in that it has a ring 1104 on which a plurality of spikes 1106 having hook tips 1108 are mounted. These spikes pass through apertures. 1112 in a base ring 1110. In one embodiment of the invention, however, base ring 1110 includes a second array of apertures 1114, through which a plurality of graft-pulling spikes 1116, having hooked tips 1118, are provided. These spikes may be mounted on a second ring (not shown) or they may be part of the delivery system.

In this connector, instead of everting graft 100 over spikes 1106, graft end 101 is distorted so that it is at least partially everted over base ring 1110, but abuts the spikes instead of being transfixed by them.

FIG. 11A shows a starting position, in which graft 100 is inserted into connector 1102, and puller spikes 1116 are bent over so that hooks 1118 are positioned to radially distort graft end 101.

FIG. 11B shows a top view of FIG. 11A.

FIG. 11C, shows the effect of pulling spikes 1116, so that hooks 1118 engage and pull back graft end 101. Spike hooks 1108 are shown in position inside a target vessel 1120.

FIG. 11D is a top view of connector 1102 in FIG. 11C, showing that portions 1122 of graft end 101, which are between spikes are pulled past spikes 1106. Portions 1124 that are adjacent spikes are pulled back to abut spikes 1108. In general, both types of portions are everted 90°, so that their intima can contact target vessel 1120. Optionally, a radial depression is formed in the base of spikes 1106, to allow portions 1124 to be pulled out more.

In FIG. 11E, spikes 1106 are pulled back(e.g., by pulling back ring 1104), so that hooks 1108 engage target vessel 1120 and the anastomosis is completed.

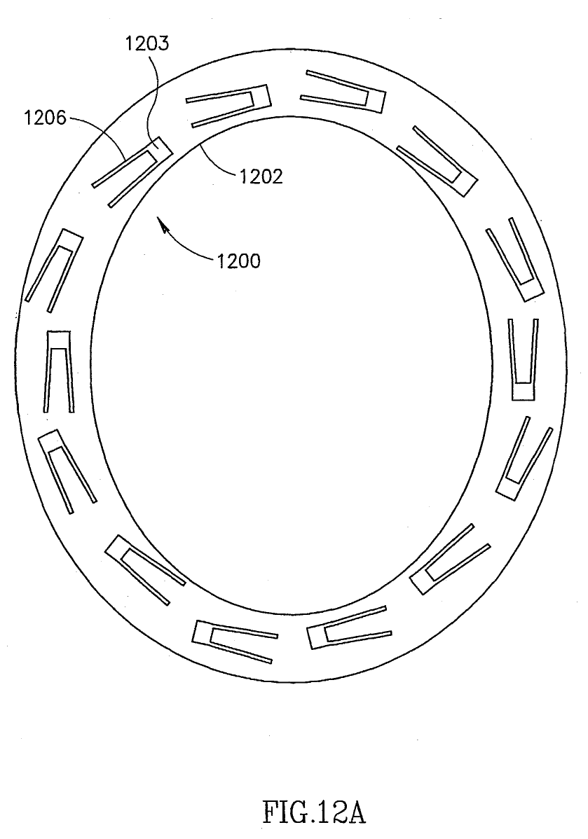

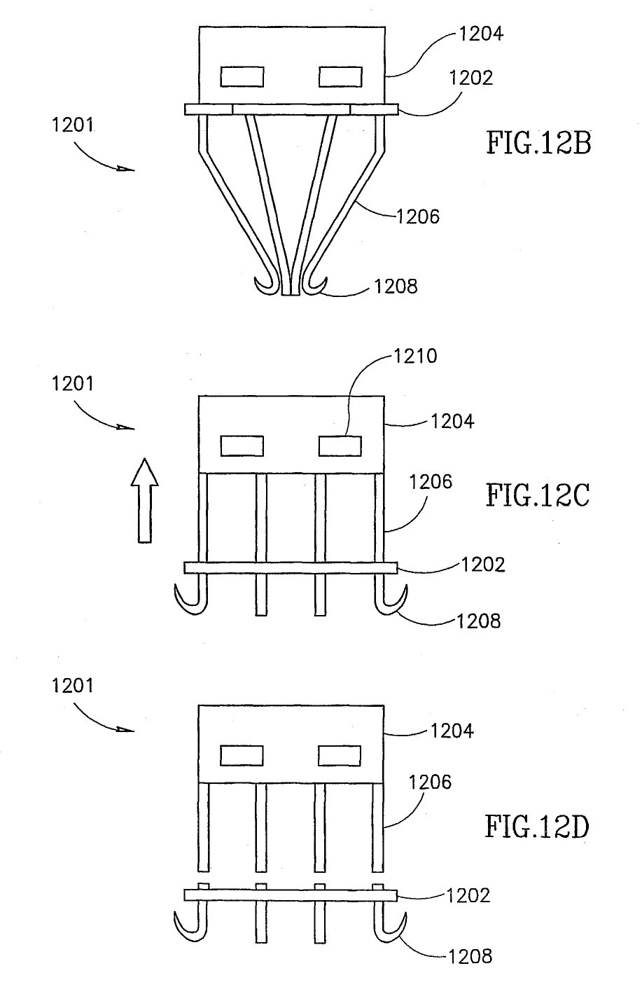

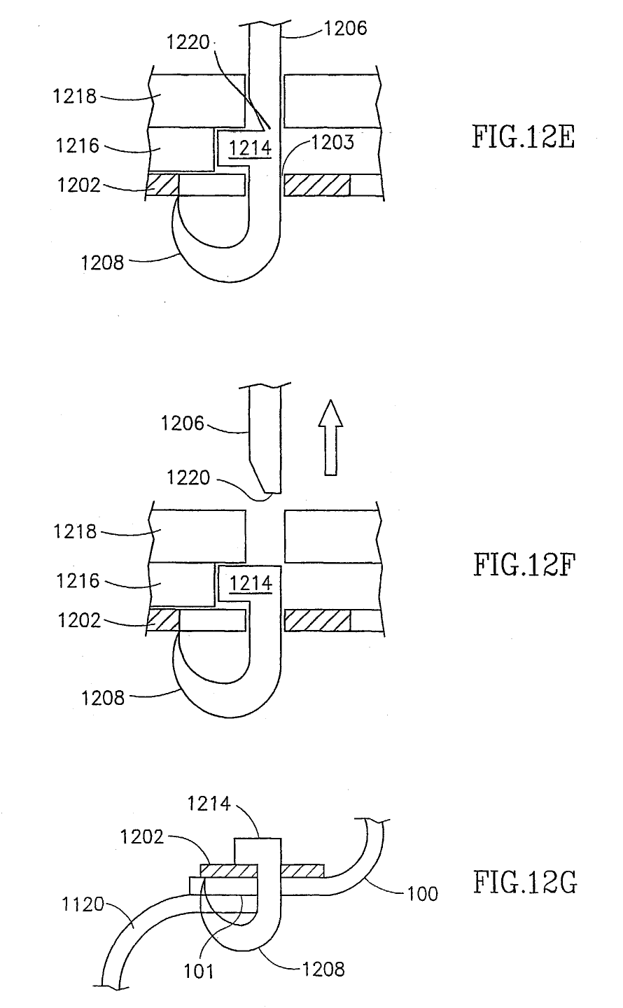

Optionally, spikes 1116 are further retracted, so that they release graft end 101 and are removed from the body. In some embodiments, spike hooks 1118 may rip through portions 1124. Alternatively or additionally, spikes 1116 are made of a bio-absorbable material. Possibly, spikes 1116 are attached to a delivery system used to deliver and deploy connector 1102 and graft 100. Alternatively, spikes 1116 are cut, so that hooks 1118 remain in the body. Alternatively, for example as shown in FIG. 12, parts of spikes 1116 are torn off.

As shown, apertures 1114 are further out radially than apertures 1112. However, they may be at a same radial distance in other designs.

In an alternative embodiment of the invention, apertures 1114 are formed in a separate ring (not shown), which is part of the delivery system (not shown). After deployment, this other ring may be removed from the body.

Alternatively or additionally to apertures 1114 and 1112 being enclosed apertures, slots or slits in ring 1110 (e.g., with openings to the outside of ring 1110) maybe provided instead.

FIG. 12A illustrates an exemplary base ring 1200 of an anastomotic connector, in accordance with an exemplary embodiment of the invention. Ring 1200 may be used for any of the connectors described above. Ring 1200 includes a base part 1202 having formed therein a plurality of apertures 1203 for allowing spikes to pass through. Optionally, each aperture includes a leaf-spring section 1206. Possibly, when a hook is pushed through aperture 1203, the hook pushes the leaf-spring aside.

FIGS. 12B-12D illustrate a process of deploying a connector in which part of the connector is removed, in accordance with an exemplary embodiment of the invention. FIG. 12B shows a connector 1201 having a base ring 1202, for example as in FIG. 12A and a plurality of spikes 1206, having hook-tips 1208, mounted on a ring 1204.

In use, after graft 100 is mounted on spikes 1206, for example using one of the methods described above, hooks 1208 are placed into a target blood vessel, such as vessel 1120 (FIG. 11C). Ring 1204 is then retracted (FIG. 12C), for example by engaging a plurality of apertures 1210 formed therein, so that spikes 1206 and hooks 1208 are retracted and seal the anastomosis (Se also FIGS. 11A-11E). In FIG. 12D, ring 1204 and most of the length of spikes 1206 is cut off of hooks 1208. Optionally, spikes 1206 are torn, at a location that is pre-weakened for such tearing. Such weakening can be, for example, by thing or holing the connector or by chemical and/or heat treatment. In an exemplary embodiment of the invention, the weakening is formed at a distance that allows the connector to connect two vessels and, optionally, means for locking the hook portion to the ring.

FIGS. 12E-12G illustrate the effect of the process of FIGS. 12B-12D, on a single spike of the connector. FIG. 12E shows a spike 1206 that includes a weakening 1220. Optionally, spike 1206 includes an extension 1214. In an exemplary embodiment of the invention, extension 1214 is used to prevent spike 1206 from falling off ring 1202, through aperture 1203. Alternatively or additionally, extension 1214 prevents retraction of hook 1208 while tearing spike 1206.

In an exemplary embodiment of the invention, a stopper 1218, for example a ring, is provided to prevent hooks 1208 from retracting during the tearing. Such a stopper may be urged against extension 1214. Alternatively or additionally, the stopper may engage the spike, for example, by clamping on it. An optional spacer 1216 may be provided to couple stopper 1218 to ring 1202. Optionally, the clamping crimps and/or partially cuts spike 1206, so that the weakening is caused or exacerbated by the crimping.

In FIG. 12F, spike 1206 is retracted, while extension 1214 is held, so spike 1206 is torn at weakening 1220.

FIG. 12G, shows the final completed anastomosis between graft 100 and target vessel 1120 (for a single hook 1208).

Alternatively or additionally to providing an extension 1214, spikes 1206 may be pre-stressed (e.g., be super-elastic or have shape memory), so that the tom part of the spike folds back over to fold back over ring 1202. Alternatively or additionally, the end of the spike is bent over.

It will be appreciated that the above described methods and devices of vascular manipulation may be varied in many ways, including, changing the order of steps, which steps are performed inside the body and which outside, the order of making the anastomosis connections, the order of steps inside each anastomosis and the exact materials used for the anastomotic connectors. Further, in the mechanical embodiments, the location of various elements may be switched, without exceeding the spirit of the disclosure, for example, switching the moving elements for non-moving elements where relative motion is required. In addition, a multiplicity of various features, both of methods and of devices have been described. It should be appreciated that different features may be combined in different ways. In particular, not all the features shown above in a particular embodiment are necessary in every similar exemplary embodiment of the invention. Further, combinations of the above features, from different described embodiments are also considered to be within the scope of some exemplary embodiments of the invention. In addition, some of the features of the invention described herein may be adapted for use with prior art devices, in accordance with other exemplary embodiments of the invention. The particular geometric forms used to illustrate the invention should not be considered limiting the invention in its broadest aspect to only those forms, for example, where a circular lumen is shown, in other embodiments an oval lumen may be used.

Also within the scope of the invention are surgical kits which include sets of medical devices suitable for making a single or a small number of anastomosis connections and/or eversions. Measurements are provided to serve only as exemplary measurements. for particular cases, the exact measurements applied will vary depending on the application. When used in the following claims, the terms “comprises”, “comprising”, “includes”, “including” or the like means “including but not limited to”.

It will be appreciated by a person skilled in the art that the present invention is not limited by what has thus far been described. Rather, the scope of the present invention is limited only by the following claims.

1. A method of mounting a graft on a spiked connector, in which the tips of the spikes define a periphery, comprising:
placing the graft, in said periphery, between tips of said spikes and a piercable element;
impaling said piercable element on said spikes, such that the graft is also impaled on said spikes; and
removing said piercable element from said spikes.

2. A method according to claim 1, comprising, pulling back impaled parts of said graft to a side of the spikes opposite said tips.

3. A method according to claim 1, wherein said piercable element is expandable, the method comprising inflating said expandable element.

4. A method according to claim 3, wherein said expansion impales said element on said spikes.

5. A method according to claim 3, wherein said expansion at least partially everts said graft.

6. A method according to claim 1, wherein said impaling comprises advancing said spike tips towards said graft.

7. A method according to claim 1, wherein said impaling comprises releasing said spike tips to advance towards said graft.

8. A method according to claim 1, comprising bending said spike tips into hooks after said impaling.

9. A method according to claim 1, comprising forming said spike tips into hooks before said impaling.

10. A method of mounting a graft on a spiked connector, comprising:
placing the graft between tips of said spikes and an element having a general outer perimeter;
advancing said spikes relative to said element such that said spikes penetrate said graft and penetrate said perimeter; and
removing said element.

11. A method according to claim 10, wherein said element is formed of a hard material and includes a plurality of depressions to receive said spikes.

12. A method according to claim 11, wherein said spikes are pre-bent and wherein said depressions guide an unbending of said spikes.

13. A method according to claim 10, wherein said element is piercable by said spikes.

14. A method according to claim 10, wherein said element is expandable.

15. A method according to claim 10, wherein said element is non-expandable.

16. A method according to any of claims 10-15, wherein advancing said spikes comprises advancing spikes that are adapted for engaging a target vessel of the anastomosis.

17. A method of mounting a graft on a connector having a plurality of spikes arranged around a central opening, said spikes having radially outward pointing hooks, comprising:
folding said spikes such that said hooks point inward into said central opening and define a periphery between them;
inserting a graft into said periphery;
advancing said hooks relative to said graft, to penetrate said graft; and
repositioning said hooks to point outward.

18. A method according to claim 17, wherein advancing said hooks comprises releasing said spikes.

19. A method according to claim 17, wherein advancing said hooks comprises moving said spikes.

20. A method according to claim 17, wherein advancing said hooks relative to said graft comprises radially expanding said graft towards said hooks.

21. A method according to claim 17, comprising, inserting a contra mandrel in said graft, to limit an advance of said hooks.

22. A method according to claim 21, wherein said contra mandrel guides said repositioning.

23. A method according to any of claims 17-22, wherein repositioning said hooks comprises unfolding said spikes.

24. A method of mounting a graft on a connector having a plurality of spikes arranged around a central opening, said spikes having radially outward pointing hooks, comprising:
inserting a graft into a periphery defined by forward ends of said spikes, in a first direction;
folding back a tip of said graft to cover said hooks; and
pulling back said graft, in a direction opposite said first direction, such that said hooks engage said folded part of said graft.

25. A method according to claim 24, wherein pulling back said graft comprises sharply pulling on said graft.

26. A method according to claim 24, comprising advancing said engaged part of said graft towards a base of said spikes, in said opposite direction.

27. A method according to any of claims 24-26, wherein said spikes define a cone, with said hooks at an apex of said cone.

28. A method of forming spike tips of an anastomotic connector, into hooks for engaging blood vessels, comprising:
providing an anastomosis connector having a plurality of straight spikes having tips;
first bending said tips at a first angle, using a first mandrel; and
second bending a furtherly distal portion of said tips using a second mandrel, to form a hook shape of said tips.

29. A method according to claim 28, comprising mounting a graft on said spikes of said connector prior to said first bending.

30. A method according to claim 28 or claim 29, wherein said first and said second mandrel have different outside diameters.

31. An anastomotic connector, comprising:
a base ring; and
a plurality of hooked spikes that pass through said base ring, which spikes include a weakening adjacent said hooks,
wherein a position of said weakening on said spikes is located on said spike so that the spikes can be torn off at said weakening by retracting said spikes relative to said hooks.

32. A connector according to claim 31, wherein said spikes include an extension perpendicular to said spike and distal of said weakening.

33. A connector according to claim 32, wherein said extension has an extent greater than apertures in said base ring through which said spikes pass.

34. A connector according to claim 32, wherein said extension is adapted to serve as a base for holding said hooks, when tearing said spikes.

35. A connector according to claim 31, wherein said spike have tips that are pre-stressed to bend when said spike is torn.

36. A connector according to claim 32, wherein said extension serves as a ratchet mechanism.

37. A connector according to claim 31, wherein said base ring comprises a plurality of apertures for said spikes.

38. A connector according to claim 37, wherein the apertures include a spring formed of said base ring.

39. An anastomotic connector, comprising:
a base ring; and
a plurality of target spikes, adapted to engage a target vessel, which target spikes pass through said ring; and
a plurality of retractable pulling spikes, having tips adapted to engage a graft placed in the lumen of said ring, and being adapted to at least partially evert a lip of said graft when said pulling spikes are retracted.

40. A connector according to claim 39, wherein said target spikes are hooked.

41. A connector according to claim 39, wherein said target spikes are inclined towards an axis of said connector.

42. A connector according to claim 39, wherein said puller spikes are provided through said base ring.

43. A connector according to claim 39, wherein said puller spikes are provided through a ring other than said base ring.

44. A connector according to claim 39, wherein said puller spikes include at least one weakened location, to facilitate tearing of said spikes.

45. A connector according to claim 39, wherein said puller spikes are adapted to be straightened by said ring, when they are retracted.

46. A method of performing an anastomosis, comprising:
engaging at least one of the vessels of a two vessel anastomosis, using a plurality of retractable spikes;
retracting said plurality of retractable spikes, to cause at least a partial eversion of said vessel.

47. A method according to claim 46, comprising, completing said anastomosis.

48. A method according to claim 46, comprising, removing said retractable spikes.

49. A method of partially everting a graft on a connector, comprising:
inserting a graft into a ring shaped anastomosis connector having a plurality of spikes; and
pulling an end of said graft radially out, so that said end abuts said spikes adjacent the spikes and extends radially out of said spikes between said spikes.

50. A method according to claim 49, wherein said pulling comprises pulling using retractable spikes.

51. A method of twisting hook-tipped spikes in a ring-type anastomotic connector so that the hooks point inward rather than outward, comprising:
arranging said spikes between two rings; and
rotating one of said rings relative to the other of said rings such that said spikes are twisted at least 120°.

52. A method according to claim 51, wherein at least one of said rings comprises a rubber ring.

53. A method according to claim 51, comprising holding said twisted spikes in a twisted configuration, during an eversion process.

54. A method of selecting an anastomotic connector for a graft, comprising:
test everting a graft on a hollow, tubular gauge having a gauge diameter; and
if said everting succeeds, selecting a connector having a size that matches said gauge diameter.

55. A method according to claim 54, wherein said selected connector is pre-loaded in a delivery system, in a sterile package.

56. A method according to claim 55, wherein said package is color-coded to match said gauge.

57. A method according to claim 54, wherein test everting comprises comprising selecting first a larger gauge for test-everting.

58. A method according to claim 54, wherein test everting comprises selecting first a smaller gauge for test-everting, first.

59. A medical graft expander, comprising:
a body having a first diameter;
a tube having a second diameter smaller than said first diameter, mounted at an end of said body, and defining an inner chamber; and
a shaft, positioned in said body and axially advancable into said chamber, said shaft having at least one section with a diameter greater than a diameter of said chamber, such that when said section is advanced into said chamber, said tube is radially expanded.

60. An expander according to claim 59, wherein said tube is made of a soft material.

61. An expander according to claim 60, wherein said tube is made of silicone.

62. An expander according to claim 59, wherein said tube is sized to be larger than an inner diameter of a graft, when the tube is expanded.

63. An expander according to claim 59, wherein said tube is sized to match an inner diameter of a graft, when the tube is expanded.

64. A graft everting method, comprising:
mounting at least an end of said graft on an expandable tube;
expanding said tube to engage and expand said graft, and
rolling back at least a portion of said end over itself.

65. A method according to claim 64, comprising, providing a tube over said graft, so said rolling back is on to said tube.

66. A method according to claim 65, wherein said tube is pre-loaded with an anastomotic connector.
